(19) United States
(12) Patent Application Publication
(10) Pub. No.: US 2005/0130187 A1
(43) Pub. Date: Jun. 16, 2005

(54) Method for identifying relevant groups of genes using gene expression profiles

(76) Inventors: Mi Young Shin, Daejeon-Shi (KR); Eun Mi Kang, Daejeon-Shi (KR); Seon Hee Park, Daejeon-Shi (KR)

Correspondence Address: JACOBSON HOLMAN PLLC, 400 SEVENTH STREET N.W., SUITE 600, WASHINGTON, DC, 20004, US

(21) Appl. No.: 10/919,284

(22) Filed: Aug. 17, 2004

(30) Foreign Application Priority Data
Dec. 13, 2003 (KR) 2003-91012

(51) Int. Cl. C12Q 1/68; G06F 19/00; G01N 33/48; G01N 33/50

(52) U.S. Cl. 435/6; 702/20

(57) ABSTRACT

Provided is a method for identifying relevant groups of genes using gene expression profiles. More particularly, it is provided a method for identifying relevant groups of genes using gene expression profiles, which analyzes the gene expression profiles obtained from microarray experiments to automatically extract seed genes of significance and identifies the relevant groups of genes based on the extracted seed genes, so that effective identification is possible regardless of the number of genes and a blind setting of initial input parameters are not required for users to readily use the method, wherein the method comprises the steps of (a) preprocessing the gene expression profiles; (b) setting the number of gene groups to be desired (k) and a input parameter(s); (c) extracting k seed genes (k=1, 2, 3, . . . ,n) based on the set input parameter(s); (d) identifying relevant groups of genes by means of the extracted seed genes; and (e) evaluating the identified relevant groups of genes.

### BACKGROUND

1. Field of the Invention

The present invention relates to a method for identifying relevant groups of genes using gene expression profiles, and more particularly, to a method for analyzing the gene expression profiles obtained from microarray experiments by automatically finding seed genes first and then identifying the relevant groups of genes based on the seed genes, so that effective identification of gene groups is possible without requiring blind parameter setting procedure to readily use the method.

2. Discussion of Related Art

In general, gene expression patterns exhibit similar properties under various environments in the case of genes having biologically similar functions or being regulated together.

Thus, methods for identifying biologically relevant gene groups have usually employed gene expression profiles, which are a collection of measurements of gene expression levels under various conditions, to find some characteristics of gene expression patterns exhibited in the expression profiles.

Such method not only allows us to characterize unknown genes from known genes belonging to the same group, but also helps us to find candidate groups of genes more likely to be biologically related. Further, it can be also used as a preprocessing step for genetic network modeling.

A method for identifying relevant gene groups using gene expression profiles (information) in the related art is as follows.

In accordance with the hierarchical clustering method entitled “cluster analysis and display of genome-wide expression patterns” to Eisen and three other inventors disclosed in the ‘Proc. Natl. Acad. Sci’, a tree-like dendrogram is generated and visualized according to a similarity degree of expression patterns between genes, and users may properly define gene groups with reference to the dendrogram.

This method has an advantage in that the users may directly and readily determine clusters from the dendrogram with reference to the gene expression patterns. On the other hand, it takes long time to build the dendrogram for large number of genes to be analyzed.

Also, in the paper disclosed in Nature Genetics entitled “Systematic determination of genetic network architecture” to Tavazoie and four other inventors, k-means clustering method is disclosed. For a user specified number of clusters (or gene groups) k, it randomly chooses initial centers of k clusters and assigns each gene to the cluster of which center is the nearest to the gene. Then, the cluster centers are refined by taking averaged expression patterns of genes belonging to each cluster. The above learning steps are iterated until the objective function is optimized.

Such method only requires the number of clusters (k) to be selected, so its simplicity and efficiency has led to wide use in many application fields, including the problem of identifying relevant groups of genes using gene expression profiles.

However, since clustering results are dependent on the randomly chosen initial centers, they can be various according to the setting of the initial centers even for the same gene expression profile.

In addition, in the US patent publication (U.S. 2002/0115070 A1) entitled “Methods and Apparatus for analyzing gene expression data” to Tamayo and three other inventors, a method for generating self-organizing maps is used to group genes exhibiting similar expression patterns.

According to this method, when a user specify a geometry structure of clusters to be generated and other parameter values necessary for learning in the beginning, the reference expression vectors of the clusters are arbitrarily chosen, and each gene is allocated to the cluster that has the most similar reference expression vector. At each time, the reference expression vectors are iteratively refined so as to well reflect the expression vectors of the allocated genes. Once the reference expression vector of each cluster is learned, each gene is allocated to the cluster of which reference expression vector is most similar to the gene, so that a final relevant group of genes may be determined.

Such method has advantages in that it is allowed to impose partial structure on the clusters and facilitates easy visualization and interpretation. However, the user may have difficulty in properly setting initial values of various parameters as well as geometrical structure.

Even though several clustering methods are employed as above for the identification of gene groups to be biologically related, we still have difficulties in doing it because of hardness to proper selection of initial parameters.

### SUMMARY OF THE INVENTION

The present invention is directed to a method for identifying relevant groups of genes using gene expression profiles, which analyzes gene expression profiles to automatically find seed genes first and then identify relevant groups of genes based on the seed genes, so that effective identification is possible without requiring a blind parameter setting procedure to readily use the method.

One aspect of the present invention is to provide a method for identifying relevant groups of genes using gene expression profiles, which comprises the steps of (a) preprocessing the gene expression profiles; (b) setting the number of gene groups to be desired (k) and a input parameter(s); (c) extracting k seed genes (k=1, 2, 3, . . . ,n) based on the set input parameter(s); (d) identifying relevant groups of genes using the extracted seed genes; and (e) evaluating the identified relevant groups of genes.

In the above-mentioned configuration, the step (c) preferably includes the sub-steps of: (c1) defining n Gaussian function (G_{i,i=1,2,3, . . . ,n}) in which their centers are defined as each gene expression vector (g_{i,i=1,2,3, . . . ,n}) and their widths are globally set as the value of input parameter (s); (c2) transforming each gene expression vector (g_{i i=1,2,3, . . . ,n}) by means of the defined Gaussian function (Gᵢ) to generate a transformed expression matrix (Φ); (c3) obtaining a permutation matrix (P) to determine k column vectors having the highest inter-independency from the generated transformed expression matrix (Φ); (c4) rearranging gene expression profiles in the order of higher independency by means of the obtained permutation matrix (P); and (c5) selecting 1^{st} to k^{th} genes from the rearranged gene expression profiles to finally determine k seed genes.

The step (c3) preferably includes the sub-steps of: (c3-1) computing singular value decomposition (SVD) of the transformed expression matrix (Φ); (c3-2) obtaining a matrix (V_{1, . . . ,k}) composed of vectors of 1^{st} to k^{th} columns of the computed right singular value matrix (V); and (c3-3) applying QR factorization to a transposed matrix of the obtained matrix (V_{1, . . . ,k}).

The step (d) preferably includes the sub-steps of: (d1) setting the extracted k seed genes (c₁,c₂, . . . ,cₖ) to a center of a cluster; and (d2) determining cluster membership (Cluster(gᵢ)) of each gene as the cluster of which center has an expression vector that is the highest relevant to that of each gene.

### BRIEF DESCRIPTION OF THE DRAWINGS

FIG. 1 shows a block configuration of an apparatus for implementing a method for identifying relevant groups of genes using gene expression profiles in accordance with one embodiment of the present invention.

FIG. 2 is a flow chart for generally explaining a method for identifying relevant groups of genes using gene expression profiles in accordance with one embodiment of the present invention.

FIG. 3 is a flow chart for explaining a step of automatically extracting a seed gene in a method for identifying relevant groups of genes using gene expression profiles in detail in accordance with one embodiment of the present invention.

FIG. 4 is a flow chart for explaining a step of identifying relevant groups of genes based on seed gene in a method for identifying relevant groups of genes using gene expression profiles in detail in accordance with one embodiment of the present invention.

FIG. 5 is a flow chart for explaining a step of identifying relevant groups of genes based on a seed gene in a method for identifying relevant groups of genes using gene expression profiles in detail in accordance with another embodiment of the present invention.

FIG. 6 shows a general format of gene expression profile obtained from a microarray experiment in accordance with one embodiment of the present invention.

FIG. 7 shows a sample gene distribution and seed genes automatically set by a method for identifying relevant groups of genes using gene expression profiles in accordance with one embodiment of the present invention.

FIG. 8 is a chart for showing experimental results obtained by a method for identifying relevant groups of genes using gene expression profiles in accordance with embodiments of the present invention.

### DETAILED DESCRIPTION OF PREFERRED EMBODIMENTS

The present invention will now be described more fully hereinafter with reference to the accompanying drawings, in which preferred embodiments of the invention are shown. This invention may, however, be embodied in different forms and should not be construed as limited to the embodiments set forth herein.

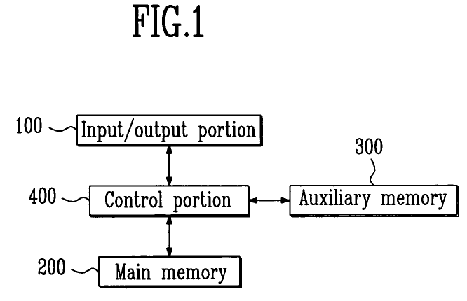

FIG. 1 shows a block configuration of an apparatus for implementing a method for identifying relevant groups of genes using gene expression profiles in accordance with one embodiment of the present invention.

As shown in FIG. 1, an apparatus for implementing a method for identifying relevant groups of genes using gene expression profiles in accordance with the present invention, is composed of an input/output portion 100 for inputting/outputting gene expression profiles and other relevant data that are necessary for external users and relevant gene group identification, main/auxiliary memories 200 and 300 for setting important seed genes using gene expression profiles obtained from the microarray experiments and for storing desired data while identifying the relevant gene groups, and a control portion 400 for controlling the main/auxiliary memories 200 and 300 and the input/output portion 100 while setting important seed genes by means of the gene expression profiles obtained from the microarray experiments and processing several calculations necessary for identifying the relevant gene groups.

In the above-mentioned configuration, the control portion 400 is preferably implemented as microprocessor, and when program including the method for identifying the relevant genes employing the gene expression profiles of the present invention is installed in the control portion 400 and the gene expression profiles are input to run the program, this program sets important seed genes, which leads to allow gene groups having biologically similar functions to be identified based on the above-mentioned configuration.

Hereinafter, the method for identifying the relevant groups of genes using gene expression profiles of the present invention having the above-mentioned configuration will be described in detail.

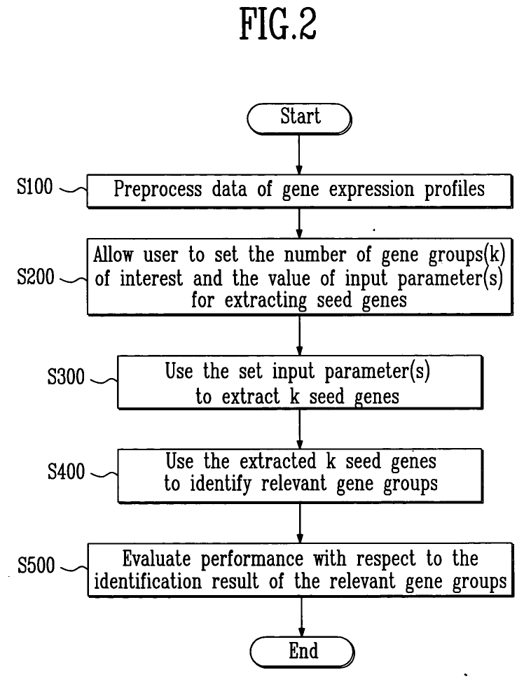

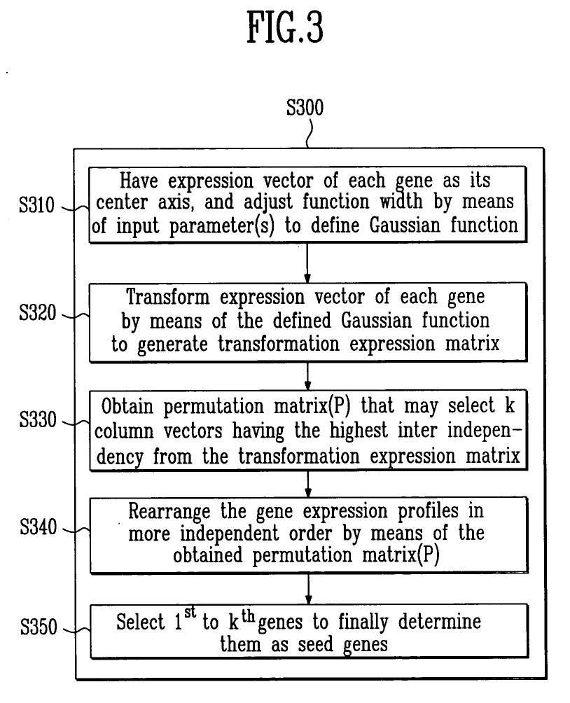

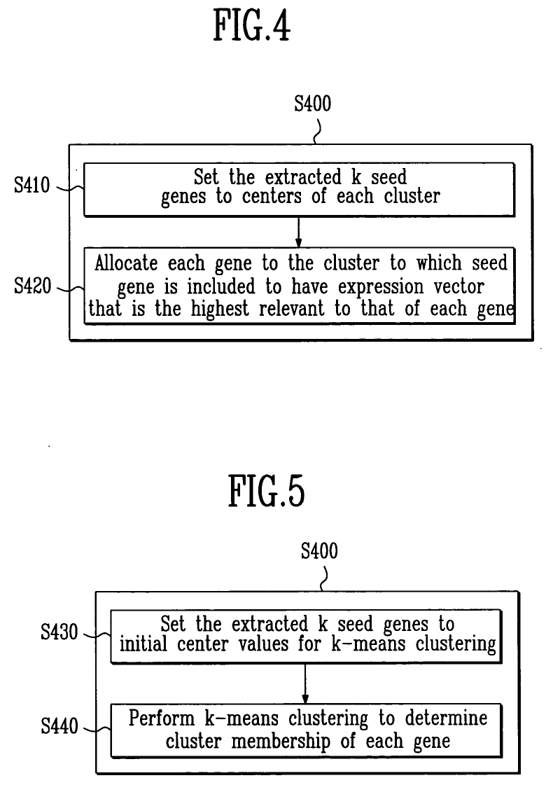

FIG. 2 is a flow chart for generally explaining a method for identifying relevant groups of genes using gene expression profiles in accordance with one embodiment of the present invention, and FIG. 3 is a flow chart for explaining a step of automatically extracting seed genes in a method for identifying relevant groups of genes using gene expression profiles in detail in accordance with one embodiment of the present invention, and FIG. 4 is a flow chart for explaining a step of identifying relevant groups of genes based on seed genes in a method for identifying relevant groups of genes using gene expression profiles in detail in accordance with one embodiment of the present invention, wherein the control portion 400 plays a major role for the above-mentioned configuration, unless specifically described.

As shown in FIG. 2 to FIG. 4, gene expression profiles are first preprocessed to facilitate identification of gene groups according to the similarity of gene expression patterns in the step S100, and the number of gene groups (k=1, 2, 3 . . . ,n) of interest and an input parameters (s) are set to extract k seed genes from the preprocessed gene expression profiles in the step S200.

Next, k seed genes are extracted using the set input parameter (s) in the step S300, and relevant groups of genes are identified using the extracted seed genes in the step S400. The extracted seed genes are taken as representative centers of k clusters to be identified, and each gene is allocated to a cluster to which the nearest seed gene is included.

The identified relevant groups of genes are evaluated in the step S500, in other words, when group allocation is completed with respect to all the genes, performance evaluation of the obtained results, viz. the identified relevant groups of genes, is performed using a cluster validation index.

In the meantime, steps S300, S400, and S500 may be repeatedly performed after the set values is changed with respect to the input parameter (s) in the steps S200, and the identification result having the most superior performance is finally selected.

In order to preprocess the gene expression profile in the step S100, genes are filtered out that have almost no difference or have no significant pattern changes over various experimental conditions.

In addition, when missing value with respect to a specific experiment of a specific gene is found, the missing value may be recovered by filtering out such gene or predicting an expected expression value by means of computational method.

In the meantime, in order to define the similarity of genes in terms of the pattern change of expression values instead of absolute expression values, a data preprocessing procedure is performed to normalize expression values by fixing a mean and a standard deviation of the expression values per gene within a constant range.

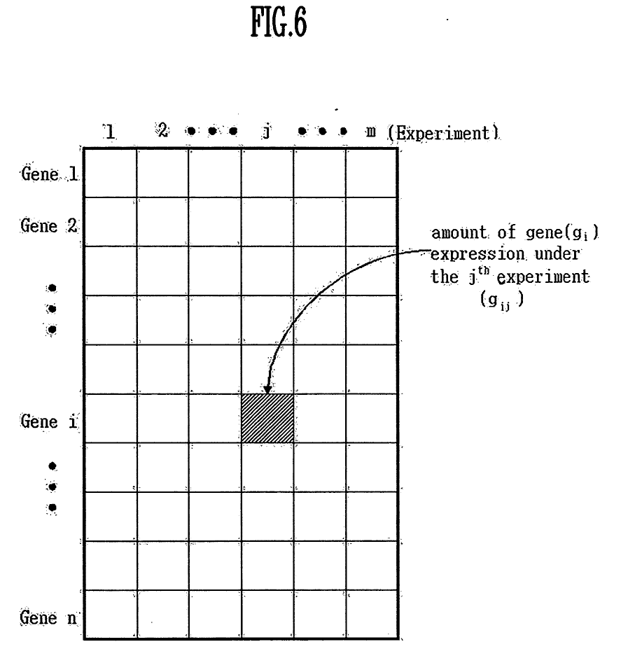

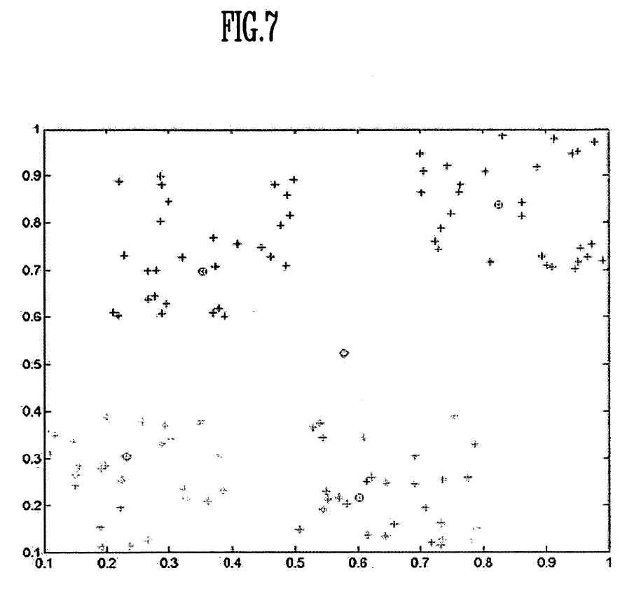

Specifically, in the case that the mean (Mean) and standard deviation (Std) of the expression values to be fixed are designated, the expression value (gᵢⱼ) under the j^{th} experimental condition of i^{th} gene may be transformed into the normalized expression value (g′ᵢⱼ) by the following equation 1 where the desired mean and standard deviation are \overline{g}ᵢ and σᵢ, respectively. (See FIG. 6)

$${g}_{ij}^{′} = \frac{Std \times ( {g}_{ij} - {\overline{g}}_{i} )}{{σ}_{i}} - Mean \qquad equation 1$$

To detail extraction procedure of seed genes in the step S300, n Gaussian functions (G_{i,i=1,2, . . . ,n}) is defined in which their widths are globally adjusted in accordance with the input parameters (s) set by the user in the step S310, and the centers are defined as gene expression vectors (g_{i,i=1,2, . . . ,n}) exhibiting expression values in response to various experimental conditions per gene.

Next, in the step S320, each gene expression vector (gᵢ) is transformed by the n Gaussian functions (Gᵢ) to thereby generate a transformed expression matrix (Φ). In this case, the configuration of the transformed expression matrix (Φ) is determined by the following equation 2.

$${Φ}_{ij} = exp ( - \frac{{‖ {g}_{i} - {g}_{j} \vert}^{2}}{2 {s}^{2}} ) \qquad equation 2$$

Next, in the step S330, when the transformed expression matrix (Φ) is generated, k column vectors are selected that have the highest inter-independency from the transformed expression matrix (Φ) so as to determine k seed genes. In other words, a permutation matrix (P) is obtained so as to determine the k column vectors that have the highest inter-independency.

By way of specific example for obtaining such permutation matrix (P), singular value decomposition (SVD) of the transformation expression matrix (Φ) is computed and a matrix (V_{1, . . . ,k}) is obtained to consist of column vectors from 1^{st} to k^{th} of right singular value matrix (V) resulted from the calculation, and QR factorization is applied to a transposed matrix of the obtained matrix (V_{1, . . . ,k}), so that the permutation matrix (P) may be readily obtained.

Next, in the step S340, the gene expression profiles are rearranged in the order of higher independency using the obtained permutation matrix (P), and 1^{st} to k^{th} genes are selected from the rearranged gene expression profiles in the step S350, so that k seed genes are finally determined.

In the meantime, to detail the procedure of identifying relevant groups of genes in the step S400, the extracted k seed genes (c₁,c₂, . . . ,cₖ) are set to centers of cluster in the step S410, cluster membership (Cluster(gᵢ)) of genes is determined as the cluster of which center is most similar expression vector to that of each gene in the step S420. In other words, the cluster membership (Cluster(gᵢ)) of each gene is determined as a cluster index of the seed gene having the expression vector which is the most similar to that of gene.

In this case, the cluster membership (Cluster(gᵢ)) of each gene (gᵢ) may be determined by the following equation 3, and the gene having the same cluster membership represents the final identification result with respect to a relevant group of genes.

$$Cluster ( {g}_{i} ) - arg {min}_{j} ‖ {g}_{i} - {c}_{2} \vert \qquad equation 3$$

FIG. 5 is a flow chart for explaining a step of identifying relevant groups of genes based on seed genes in a method for identifying relevant groups of genes using gene expression profiles in detail in accordance with another embodiment of the present invention.

As shown in FIG. 5, to detail other specific procedures of identifying the relevant groups of genes in the step S400, the k extracted seed genes are set to initial center values for k-means clustering in the step S430, and clusters are generated in response to the set initial center values to determine cluster membership of each gene by means of the generated clusters in the step S440.

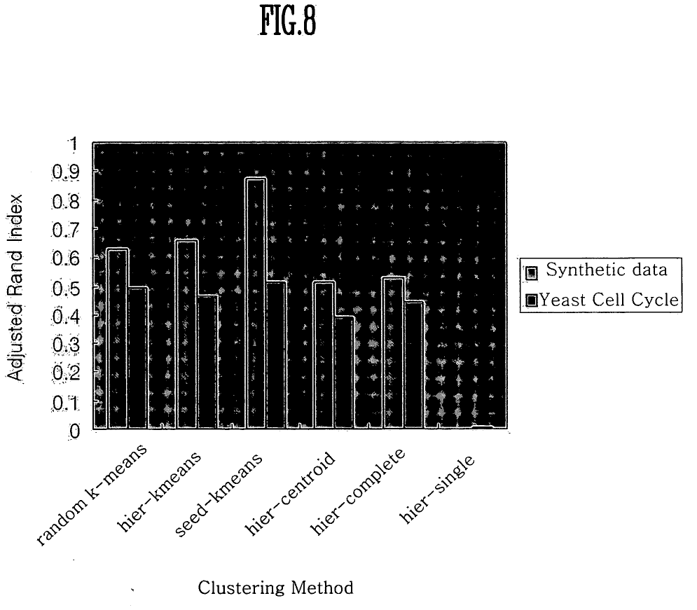

FIG. 6 shows a general format of gene expression profile obtained from a microarray experiment in accordance with one embodiment of the present invention, FIG. 7 shows the sample gene distribution and seed genes automatically set by a method for identifying relevant groups of genes using gene expression profiles in accordance with one embodiment of the present invention, and FIG. 8 is a chart for showing experimental results obtained by a method for identifying relevant groups of genes using gene expression profiles in accordance with embodiments of the present invention.

As shown in FIG. 6 to FIG. 8, typical cluster verifying index may be used to evaluate the identified relevant groups of genes in the above-mentioned step S500.

In addition, when external knowledge is available about cluster memberships, validation index such as Adjusted Rand Index may be employed to quantify the similarity degree between relevant groups of genes defined by the external criterion, and the identified relevant groups of genes.

In the meantime, when there is no external knowledge available, cluster validation index such as Figure-of-merit that is commonly used may be employed to evaluate identification result.

While the present invention has been described for the method for identifying relevant groups of genes using gene expression profiles with reference to a preferred embodiment, it should be understood that the disclosure has been made for illustrative purpose of the invention via examples and is not limited to limit the scope of the invention. And one skilled in the art can make amend and change the present invention without departing from the scope and spirit of the invention.

In accordance with the method for identifying the relevant groups of genes using gene expression profiles of the present invention as mentioned above, gene expression profiles obtained from the microarray experiment are analyzed to automatically extracted seed genes of significance, and the system may identify the relevant groups of genes based on the analysis, which allows the system to effectively identify gene groups regardless of the number of corresponding genes, and excludes the use of random initial genes, so that the consistency of the identification result may be kept and a blind setting of initial input parameters are not required to facilitate work by users.

1. A method for identifying relevant groups of genes using gene expression profiles, the method comprising the steps of:
(a) preprocessing the gene expression profiles;
(b) setting the number of gene groups to be desired (k) and a input parameter(s);
(c) extracting k seed genes (k=1, 2, 3, . . . ,n) based on the set input parameter(s);
(d) identifying relevant groups of genes using the extracted seed genes; and
(e) evaluating the identified relevant groups of genes.

2. The method as claimed in claim 1, wherein the step (a) of preprocessing the gene expression profiles so as to facilitate identifying gene groups in accordance with relevance of an expression pattern includes a sub-step of fixing a mean (Mean) and a standard deviation (Std) of an expression value per gene in a constant range and then obtaining a normalized expression value by means of the following equation
$${g}_{ij}^{′} = \frac{Std \times ( {g}_{ij} - {\overline{g}}_{i} )}{{σ}_{i}} - Mean ,$$
where gᵢⱼ represents the expression value under the j^{th} (j=1, 2, 3, . . . ,n) experimental condition of i^{th} (i=1, 2, 3, . . . ,n) gene, and \overline{g}ᵢ and σᵢ represent the mean and standard deviation of the expression value with respect to the experimental condition per gene, respectively.

3. The method as claimed in claim 1, wherein the step (c) includes the sub-steps of:
(c1) defining n Gaussian functions (G_{i,i=1,2,3, . . . ,n}) in which their widths are globally adjusted in response to an input parameter (s) set by a user and their centers are defined as an expression vector (g_{i,i=1,2,3, . . . ,n}) representing the expression value in response to various experimental conditions per gene from the gene expression profiles;
(c2) transforming the expression vector (gᵢ) per gene by means of the defined Gaussian function (Gᵢ) to generate a random transformed expression matrix (Φ);
(c3) obtaining a permutation matrix (P) to determine k column vectors having the highest inter-independency from the generated transformed expression matrix (Φ);
(c4) rearranging the gene expression profiles in an order of higher independency by means of the obtained permutation matrix (P); and
(c5) selecting 1^{st} to k^{th} genes from the rearranged gene expression profiles to finally determine k seed genes.

4. The method as claimed in claim 3, wherein the transformed expression matrix (Φ) in the step (c2) is generated by the following equation
$${Φ}_{ij} = exp ( - \frac{{‖ {g}_{i} - {g}_{j} \vert}^{2}}{2 {s}^{2}} )$$

5. The method as claimed in claim 3, wherein the step (c3) includes the sub-steps of:
(c3-1) computing singular value decomposition (SVD) of the transformed expression matrix (I);
(c3-2) obtaining a matrix (V_{1, . . . ,k}) composed of vectors of 1^{st} to k^{th} columns of the computed right singular value matrix (V); and
(c3-3) applying QR factorization to a transposed matrix of the obtained matrix (V_{1, . . . ,k}).

6. The method as claimed in claim 1, wherein the step (d) includes the sub-steps of:
(d1) setting the extracted k seed genes (c₁,c₂, . . . ,cₖ) to a center of a cluster; and
(d2) determining cluster membership (Cluster(gᵢ)) of gene with the cluster of which center has an expression vector that is the highest relevant to that of each gene.

7. The method as claimed in claim 6, wherein the cluster membership (Cluster(gᵢ)) of gene in the step (d2) is determined by the following equation
$$Cluster ( {g}_{i} ) - arg {min}_{j} ‖ {g}_{i} - {c}_{2} \vert$$

8. The method as claimed in claim 1, wherein the step (d) includes the sub-steps of:
(d3) setting the extracted k seed genes to initial center values for k-means clustering; and
(d4) generating k clusters based on the set initial center values to determine the cluster membership of each gene with the generated cluster.
